SEQUENCE LISTING (30 sequences)

SEQ ID NO: 1 - protein, 26 aa, Human HIV
  MISC_FEATURE (1)..(1): Xaa=G
XXRGDXFGXXLLFIHFXIGSXHSXIX

SEQ ID NO: 2 - protein, 28 aa, Human HIV
GGCRGDMFGCGGLLFIHFRIGSRHSRIG

SEQ ID NO: 3 - protein, 28 aa, Human HIV
GGCRGDMFGCGGLLRIHFRIGSRHSRIG

SEQ ID NO: 4 - protein, 27 aa, Human HIV
GGCRGDMFGCGGLFIHFRIGSRHSRIG

SEQ ID NO: 5 - protein, 28 aa, Human HIV
GGCRGDMFGCGGSLFIHFRIGSRHSRIG

SEQ ID NO: 6 - protein, 28 aa, Human HIV
GGCRGDMFGCGGLLFIHFKIGSRHSRIG

SEQ ID NO: 7 - protein, 29 aa, Human HIV
GGCRGDMFGCGGLLFIHFNRIGSRHSRIG

SEQ ID NO: 8 - protein, 28 aa, Human HIV
GGCRGDMFGCGGLLSRHFRIGSRHSRIG

SEQ ID NO: 9 - protein, 28 aa, Human HIV
GGCRGDMFGCGGLLSIHFRIGSRHSRIG

SEQ ID NO: 10 - protein, 28 aa, Human HIV
GGCRGDMFGCGGLLFRHFRIGSRHSRIG

SEQ ID NO: 11 - protein, 6 aa, Human HIV
RGDMFG

SEQ ID NO: 12 - protein, 28 aa, Human HIV
  MISC_FEATURE (3)..(3): Xaa=any amino acid
GGXRGDMFGXGGLLFIHFRIGCRHSRIG

SEQ ID NO: 13 - protein, 28 aa, Human HIV
  MISC_FEATURE (3)..(3): Xaa=any amino acid
GGXRGDMFGXGGLLFIFFRIGCRFSRIG

SEQ ID NO: 14 - protein, 28 aa, Human HIV
  MISC_FEATURE (3)..(3): Xaa=any amino acid
GGXRGDMFGXGGLLFIHFRIGSRHSRIG

SEQ ID NO: 15 - protein, 28 aa, Human HIV
  MISC_FEATURE (3)..(3): Xaa=any amino acid
GGXRGDMFGXGGLLRIHFRIGSRHSRIG

SEQ ID NO: 16 - protein, 27 aa, Human HIV
  MISC_FEATURE (3)..(3): Xaa=any amino acid
GGXRGDMFGXGGLFIHFRIGSRHSRIG

SEQ ID NO: 17 - protein, 28 aa, Human HIV
  MISC_FEATURE (3)..(3): Xaa=any amino acid
GGXRGDMFGXGGSLFIHFRIGSRHSRIG

SEQ ID NO: 18 - protein, 28 aa, Human HIV
  MISC_FEATURE (3)..(3): Xaa=any amino acid
GGXRGDMFGXGGLLFIHFKIGSRHSRIG

SEQ ID NO: 19 - protein, 28 aa, Human HIV
  MISC_FEATURE (3)..(3): Xaa=any amino acid
GGXRGDMFGXGGLLFIHFRIGSRHSRIG

SEQ ID NO: 20 - protein, 28 aa, Human HIV
  MISC_FEATURE (3)..(3): Xaa=any amino acid
GGXRGDMFGXGGLLSRHFRIGSRHSRIG

SEQ ID NO: 21 - protein, 28 aa, Human HIV
  MISC_FEATURE (3)..(3): Xaa=any amino acid
GGXRGDMFGXGGLLSIHFRIGSRHSRIG

SEQ ID NO: 22 - protein, 28 aa, Human HIV
  MISC_FEATURE (3)..(3): Xaa=any amino acid
GGXRGDMFGXGGLLFRHFRIGSRHSRIG

SEQ ID NO: 23 - protein, 28 aa, Human HIV
  MISC_FEATURE (3)..(3): Xaa=any amino acid
GGXRGDMFGXGGLLFIHFRIGSRHSRIG

SEQ ID NO: 24 - protein, 28 aa, Human HIV
GGCRADMFGCGGLLFIHFRIGSRHSRIG

SEQ ID NO: 25 - protein, 28 aa, Human HIV
GGCRGDMFGCGGLLFIHFAIGSRHSAIG

SEQ ID NO: 26 - protein, 27 aa, Human HIV
RKKRRQRRRGGLLFIHFRIGSRHSRIG

SEQ ID NO: 27 - protein, 16 aa, Human HIV
LLFIHFRIGSRHSRIG

SEQ ID NO: 28 - protein, 12 aa, Human HIV
GGCRGDMFGCGG

SEQ ID NO: 29 - protein, 12 aa, Human HIV
GGCRADMFGCGG

SEQ ID NO: 30 - protein, 12 aa, Human HIV
GGCRGDMFGCGG

(19) United States
(12) Patent Application Publication
(10) Pub. No.: US 2008/0171697 A1
(43) Pub. Date: Jul. 17, 2008

(54) Peptides Having For Example Antiangiogenic Activity and Applications Thereof In Therapeutics

(76) Inventors: Etienne Jacotot, Paris (FR); Annie Borgne-Sanchez, Paris (FR); Sylvie Dupont, Paris (FR); Dominique Rebouillat, Paris (FR)

Correspondence Address: NIXON & VANDERHYE, PC, 901 NORTH GLEBE ROAD, 11TH FLOOR, ARLINGTON, VA, 22203, US

(21) Appl. No.: 10/573,576

(22) PCT Filed: Sep. 24, 2004
(86) PCT No.: PCT/FR04/02422

§ 371 Date: Nov. 2, 2006

(30) Foreign Application Priority Data
Sep. 25, 2003 (FR) 0311270

(51) Int. Cl. A61K 38/16; C07K 14/00

(52) U.S. Cl. 514/9; 530/317

(57) ABSTRACT

The invention relates to cyclised peptides corresponding to sequence SEQ ID N^{o}1: X₁X₂RGDX₃FGX₄X₅LLFIHFX₆IGSX₇HSX₈IX₉, wherein: the letters without any numerical index correspond to amino acids defined by the single-letter international code; X₁ is G or GG having an amino-terminal end which may or may not be free; X₂ is either a C, in which case X₂=X₄, the two Cs being connected by a disulphide bridge, or X₂ is capable of forming a lactam bridge with X₄, either X₂ or X₄ being an amino acid bearing an acid group, such as A or D, and the other bearing an amino function such as Q, N; X₃ is either an M pattern or a norleucine pattern; X₅ is one or several di-, tri-, or tetra-peptide patterns comprising G or a combination of G and S, or X₅ is a C pattern having a side-chain which serves as a covalent linkage point with a 3-nitro-2-pyridinesulphenyl group which is located at the N-terminal end of the next amino acid (L); X₆ is either an R pattern or a K pattern; X₇ is either an R pattern or a K pattern; X₈ is either an R pattern or a K pattern; and X₉ is an aliphatic amino acid (such as G or A) having an amide C-terminal end. The inventive peptides can be used as active ingredients in medicaments, for example, for the treatment of pathologies linked to hypervascularisation.

The invention relates to peptides having, in particular, an antiangiogenic activity and to the applications thereof in therapeutics.

The research carried out by the inventors concerning therapeutically active peptides has led them to develop constructs which have proved to be of great interest with regard to their antiangiogenic properties.

The invention is therefore aimed at such peptides and at taking advantage of their therapeutic properties for developing medicaments. It is thus aimed at the pharmaceutical compositions containing these peptides as active ingredient. It is also aimed at the use of these peptides for producing medicaments having an antiangiogenic effect, for the treatment of pathologies associated with hypervascularization.

The peptides according to the invention are characterized in that they are cyclized peptides corresponding to the sequence SEQ ID No 1: X₁X₂RGDX₃FGX₄X₅LLFIHFX₆IGSX₇HSX₈IX₉ in which:
the letters without any numerical index correspond to amino acids defined by the single-letter international code,
X₁ is either a G or a GG, the amino-terminal end of which is free, alkylated, acylated, or in particular acetylated, or contains a labeling group, such as the biotinyl group,
X₂ is either a C, in which case X₂=X₄, the two Cs then being connected by a disulfide bridge, or X₂ is capable of forming a lactam bridge with X₄, one of X₂ or X₄ being an amino acid bearing an acid group, such as A or D, the other bearing an amino function, such as Q or N,
X₃ is either an M motif or a norleucine motif,
X₅ is either a motif, or a succession of two di-, tri- or tetrapeptide motifs composed of G or a combination of G and of S, such as GG, GGG, GGGG, GGS, GGGS or GGSGGS, or else X₅ is a C motif, the side chain (thiol function) of which serves as a point for covalent bonding with a 3-nitro-2-pyridinesulfenyl group (Npys; Drijfhout et al., 1988 Int J Peptide protein Res, 32: 161-166) located on the N-terminal end of the next amino acid (L),
X₆ is either an R motif or a K motif,
X₇ is either an R motif or a K motif,
X₈ is either an R motif or a K motif,
X₉ is an aliphatic amino acid (such as G or A), the C-terminal end of which is amidated.

These peptides contain from 25 to 35 amino acids.

A peptide of this type corresponds to the sequence SEQ ID No 2: GG*CRGDMFG*CGGLLFIHFRIGSRHSRIG (*indicates a disulfide bridge connecting the two C motifs).

Other peptides are as defined above and have an alkylated group at their N-terminal end.

In even other peptides, one or more amino acids are replaced with their dextrorotary form (_{D}aa).

Other peptides according to the invention correspond to SEQ ID No 1 above, but contain one or more peptide bonds so as to form bioisosters. Mention will, for example, be made of the reduction of an amide bridge to —CH₂NH—, or a retro-inverso reaction, as defined by Goodman and Ro (1995, in Burger's Medicinal Chemistry, Fifth ed vol. 1 pages 803-861, edited by M E Wolff).

As variants of the peptide of sequence SEQ ID No 2 exposing the RGD motif via a disulfide bridge between two cysteines, mention will be made of the peptides of sequences SEQ ID No 3 to 10:

| | |
|---|---|
| SEQ ID No 3: | GG*CRGDMFG*CGGLLRIHFRIGSRHSRIG |
| SEQ ID No 4: | GG*CRGDMFG*CGG-LFIHFRIGSRHSRIG |
| SEQ ID No 5: | GG*CRGDMFG*CGGSLFIHFRIGSRHSRIG |
| SEQ ID No 6: | GG*CRGDMFG*CGGLLFIHFKIGSRHSRIG |
| SEQ ID No 7: | GG*CRGDMFG*CGGLLFIHF^{N}RIGSRHSRIG |

(^{N}R representing an N-alkylarginine motif)

| | |
|---|---|
| SEQ ID No 8: | GG*CRGDMFG*CGGLLSRHFRIGSRHSRIG |
| SEQ ID No 9: | GG*CRGDMFG*CGGLLSIHFRIGSRHSRIG |
| SEQ ID No 10: | GG*CRGDMFG*CGGLLFRHFRIGSRHSRIG |

Other peptides of the invention contain a sequence SEQ ID No 11: X-R-G-D-M-F-GX′ exposing the RGD motif via a lactam bridge between the amino acids X (X)—C—O—NH—(X′), X and X′ being amino acids such that one bears an acid group and the other bears an amine.

Preferred peptides of this group correspond to the sequences SEQ ID No 12 to SEQ ID No 23:

| | |
|---|---|
| SEQ ID No 12: | GGXRGDMFGX′GGLLFIHFRIGCRHSRIG |
| SEQ ID No 13: | GGXRGDMFGX′GGLLFIFFRIGCRFSRIG |
| SEQ ID No 14: | GGXRGDMFGX′GGLLFIHFRIGSRHSRIG |
| SEQ ID No 15: | GGXRGDMFGX′GGLLRIHFRIGSRHSRIG |
| SEQ ID No 16: | GGXRGDMFGX′GG-LFIHFRIGSRHSRIG |
| SEQ ID No 17: | GGXRGDMFGX′GGSLFIHFRIGSRHSRIG |
| SEQ ID No 18: | GGXRGDMFGX′GGLLFIHFKIGSRHSRIG |
| SEQ ID No 19: | GGXRGDMFGX′GGLLFIHF^{N}RIGSRHSRIG |

(NR representing an N-alkylarginine motif)

| | |
|---|---|
| SEQ ID No 20: | GGXRGDMFGX′GGLLSRHFRIGSRHSRIG |
| SEQ ID No 21: | GGXRGDMFGX′GGLLSIHFRIGSRHSRIG |
| SEQ ID No 22: | GGXRGDMFGX′GGLLFRHFRIGSRHSRIG |
| SEQ ID No 23: | GGXRGDMFGX′GGLLFIHFRIGSRHSRIG |

Said sequences can be modified, i.e. can correspond to the native peptide but contain one or more different acids that are chemically modified, provided that these modifications do not affect the desired function. Mention will in particular be made of the replacement of Met with nor-Leu, and Arg with N-alkyl Arg, which makes it possible in particular to stabilize the construct. These modifications also comprise an acyl, in particular an acetyl, group in the N-terminal position. The peptides of the invention are also characterized in that they induce apoptosis in human endothelial cells expressing αVβ3 receptors.

They are also advantageously characterized in that they undergo endocytosis by human endothelial cells expressing αVβ3 receptors, localize in the mitochondrial compartment and exert a mitochondriotoxic effect.

When a peptide as developed above, or as defined above, is brought into contact with endothelial cells, specific recognition of the αVβ3 integrins at the surface of the endothelial cells is observed, which allows endocytosis of the chimeric peptide. Once internalized, the peptide localizes transiently in the lysosomes, as shown in confocal microscopy, and gradually becomes distributed within the mitochondrial compartment.

It will be noted that the specificity of the peptides of the invention results from the addition of the mitochondrial toxic part to integrin ligands so as to exert a toxicity via the mitochondrial toxicity pathway, the integrin ligands being present for the purposes of targeting and themselves having no angiostatic activity.

As illustrated by the examples given hereinafter, the treatment of human primary endothelial cells with doses of peptides of the order of one micromolar results in dissipation of the mitochondrial transmembrane potential (Δψm), in the release of mitochondrial cytochrome c, in the exposure of phosphatidylserine and in the condensation of nuclear chromatin.

These peptide constructs have the advantage of a lack of toxicity on αVβ3-negative cells.

The invention is therefore also aimed at taking advantage of these properties for selectively inducing PMM and apoptosis in angiogenic endothelial cells in the context of therapeutic strategies, in particular anticancer strategies, or the treatment of arthritis or of diabetic retinopathy.

The pharmaceutical compositions according to the invention are characterized in that they contain a therapeutically effective amount of at least one peptide, as defined above, in combination with a pharmaceutically acceptable vehicle.

These compositions are advantageously in the pharmaceutical forms suitable for their administration by injection.

Mention will in particular be made of injectable solutions for intravenous administration.

The invention is also aimed at the use of peptide constructs as defined above, for producing antiangiogenic medicaments for the treatment of pathologies due to hypervascularization.

Mention will in particular be made of the treatment of solid tumors such as pulmonary tumors, adenomas, melanomas, prostate cancer, breast cancer, colon cancer, pancreatic cancer or osteosarcomas. The invention also applies to the treatment of diabetic retinopathies and of arthritis.

The dosages of the administration forms and the treatments will be determined by those skilled in the art according to the pathology to be treated and to the patient's condition.

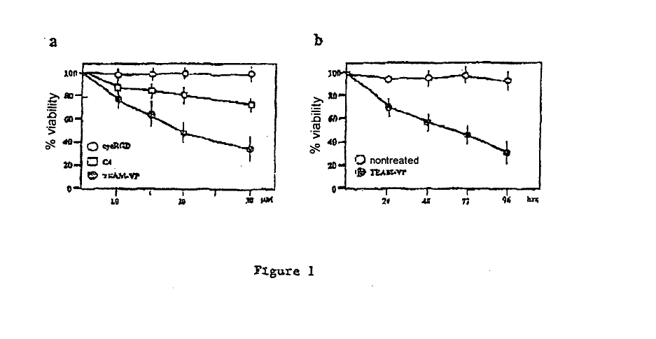

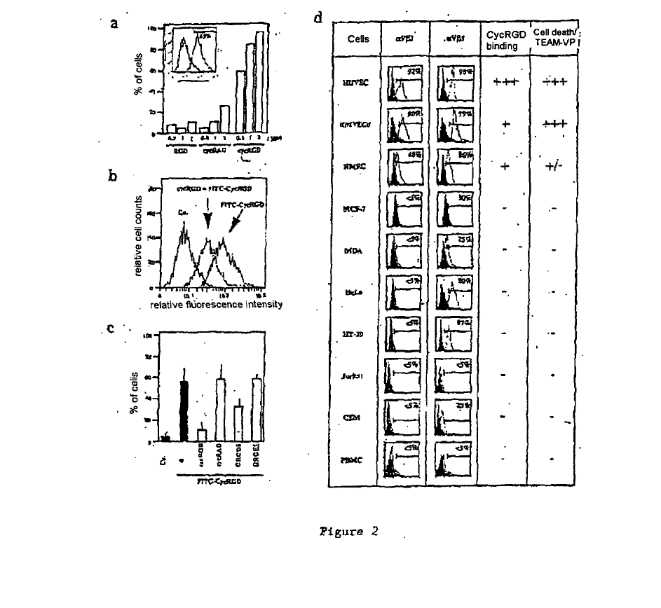

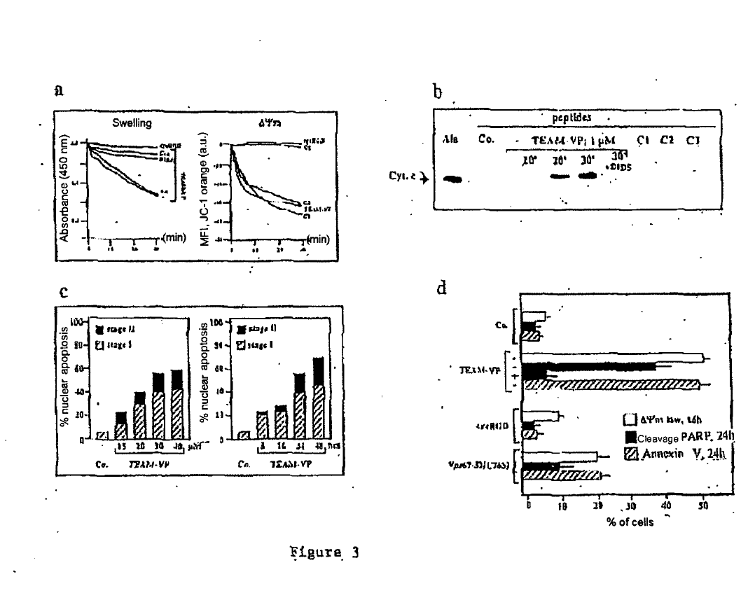

Other characteristics and advantages of the invention will be given in the examples which follow and which refer to the construct SEQ ID No 2 (hereinafter referred to as TEAM-VP) SEQ ID No 2: GG*CRGDMFG*C-GG-LLFIHFRIGSRHSRIG-amide with or without biotin, “1*” indicating a cyclization by formation of a disulfide bridge. Reference will be made to FIGS. 1 to 3, which represent, respectively,
FIG. 1, analysis of the cytotoxicity on endothelial cells,
FIG. 2, recognition of the CycRGD motif by αVβ3 integrins,
FIG. 3, the effects of TEAM-VP on isolated mitochondria and HUVEC cells.

### EXAMPLES

### Analysis of the Cytotoxicity of TEAM-VP On Endothelial Cells

a. The HUVEC cells were incubated for 24 h with 5-30 μm of peptide CycRGD, LLFIHFRIGSRHSRIG-amide (C4) or TEAM-VP, and then labeled with 7-AAD and analyzed by flow cytometry (FIG. 1a).

b. The HUVEC cells were incubated for 24, 48, 72 and 96 h with 15 μm of TEAM-VP and then labeled with 7-AAD and analyzed by flow cytometry (FIG. 1b).

### Recognition of the CycRGD Motif By αVβ3 Integrins

a. Analysis of Cell Binding

The HUVEC cells were incubated for 45 min at ambient temperature with the following peptides: GGCRGDMFGCGG-amide (linear RGD), GG*CRADMFG*CGG-amide (CycRAD) and GG*CRGDMFG*CGG-amide (CycRGD) (0.5 to 2 μm) labeled with FITC, and were analyzed by flow cytometry (FIG. 2a).

b. Chasing Off the CycRGD Peptide With Said Peptide

The HUVEC cells were or were not preincubated for 30 min at ambient temperature with 200 μM of nonlabeled CycRGD peptide before the addition of FITC-CycRGD peptide (10 μM) for 45 min., and were analyzed by flow cytometry (FIG. 2b).

c. Competition For the Integrin Sites

The HUVEC cells were or were not preincubated for 30 min at ambient temperature with 25 μM of peptide CycRGD, CycRAD, GRGDS and GRGES before the addition of FITC-CycRGD peptide (0.5 μM), and were then analyzed by flow cytometry (FIG. 2c).

d. Correlation Between Expression of Integrins, Binding And Toxicity of the Peptides

HUVEC, HMVECd, MCF-7, MDA, HeLa, HT-29, Jurkat, CEM and PBMC cells were labeled with antibodies directed against the αVβ3 and αVβ5 integrins and were analyzed by flow cytometry. The binding of the CycRGD peptide and the induction of apoptosis by TEAM-VP on the various cell types were measured (FIG. 2d).

+ Study of the Peptide Entry Process

The FITC-CycRGD and TEAM-VP (FITC) peptides enter the HUVECs and colocalize with dextran beads (cotreatment for 5 h). The entry of TEAM-VP and of the dextran beads is inhibited by treatment with sodium azide+deoxyglucose, indicating entry of the peptide by endocytosis. No entry of the FITC-CycRAD peptide into HUVECs, nor entry of the FITC-CycRGD peptide into HeLas is observed.

+ Intracellular Routing of TEAM-VP

HUVECs treated with TEAM-VP for 8, 24 and 32 h are observed using a confocal microscope.

TEAM-VP visualized with Streptavidin-Texas Red codistributes with the lysosomes (anti-Lamp2-FITC) at 8 h of treatment and would appear to leave these organelles at 24 h. TEAM-VP visualized with Streptavidin-FITC partially codistributes with the mitochondria (anti-VDAC) at 24 h and totally codistributes at 32 h. No codistribution with the Golgi apparatus (anti-Golgin) is observed throughout the treatment.

### Effects of TEAM-VP On Isolated Mitochondria And HUVEC Cells

a. Effect On Isolated Mitochondria

Induction of Mitochondrial Swelling

The isolated mitochondria were incubated with the CycRGD or TEAM-VP peptide in the presence or absence of bongkrekic acid (BA, 50 μM), of cyclosporin A (CsA, 10 μM) and of DIDS (8 μM).

Induction of the Drop In Mitochondrial Membrane Potential

The isolated mitochondria were incubated with 1 μM of TEAM-VP or its controls (C1, C2, C3), labeled with JC-1 and analyzed by flow cytometry (FIG. 3a),

| | |
|---|---|
| SEQ ID No 24: | C1 = GG*CRADMFG*CGGLLFIHFRIGSRHSRIGamide |
| SEQ ID No 25: | C1 = GG*CRGDMFG*CGGLLFIHFAIGSRHSAIGamide |
| SEQ ID No 26: | C3 = RKKRRQRRRGGLLFIHFRIGSRHSRIGamide |

b. Release of Cytochrome C

The isolated mitochondria were incubated with alamethicin (5 ug/ml) or TEAM-VP (10 μM) and the supernatant was analyzed by Western blotting with an anti-cytochrome c (FIG. 3b).

c. Analysis of Nuclear Apoptosis

The HUVEC cells treated with TEAM-VP (15-40 μM) in the presence or absence of caspase inhibitor for 8, 16, 24 and 48 h were labeled with Hoechst and observed under an inverted microscope. The percentages of cells exhibiting intact nuclei, of stage I or stage II, are reported (FIG. 3c).

d. Induction of the Drop In Mitochondrial Membrane Potential And Exposure of Phosphatidylserines In Cellula

The HUVEC cells were incubated for 24 h with 15 μM of TEAM-VP, CycRGD and C4 peptide and then labeled with JC-1 or with an anti-PARP or with annexin-V-FITC, and analyzed by flow cytometry (FIG. 3d).

+ Cytochrome C Release In Cellula

Release of cytochrome C observed by microscopy after 24 h of treatment with TEAM-VP (10 μM) and double labeling of the fixed cells with an anti-cytochrome c and an anti-VDAC.

+ Drop In Mitochondrial Membrane Potential In Cellula

Drop in potential observed under the microscope after JC-1-labeling of the HUVEC cells treated for 16-24 h with 15 μM of TEAM-VP peptide.

Animal Models

The animal models used to determine the effectiveness of the products correspond to those conventionally used (see in particular Kisher et al., 2001 Cancer Research 61:7669-7674, Galaup et al., 2003 Mol Therapy 7:731-740).

1. A peptide having, in particular, an antiangiogenic activity, characterized in that it is a cyclized peptide corresponding to the sequence
SEQ ID No 1: X₁X₂RGDX₃FGX₄X₅LLFIHFX₆IGSX₇HSX₈IX₉ in which:
the letters without any numerical index correspond to amino acids defined by the single-letter international code,
X₁ is either a G or a GG, the amino-terminal end of which is free, alkylated, acylated, or in particular acetylated, or contains a labeling group, such as the biotinyl group,
X₂ is either a C, in which case X₂=X₄, the two Cs then being connected by a disulfide bridge, or X₂ is capable of forming a lactam bridge with X₄, one of X₂ or X₄ being an amino acid bearing an acid group, such as A or D, the other bearing an amino function, such as Q or N,
X₃ is either an M motif or a norleucine motif,
X₅ is either a motif, or a succession of two di-, tri- or tetrapeptide motifs composed of G or a combination of G and of S, such as GG, GGG, GGGG, GGS, GGGS or GGSGGS, or else X₅ is a C motif, the side chain (thiol function) of which serves as a point for covalent bonding with a 3-nitro-2-pyridinesulfenyl group located on the N-terminal end of the next amino acid (L),
X₆ is either an R motif or a K motif,
X₇ is either an R motif or a K motif,
X₈ is either an R motif or a K motif,
X₉ is an aliphatic amino acid (such as G or A), the C-terminal end of which is amidated.

2. The peptide as claimed in claim 1, characterized in that it corresponds to the sequence
SEQ ID No 2: GG*CRGDMFG*CGGLLFIHFRIGSRHSRIG (*indicates a disulfide bridge connecting the two C motifs).

3. The peptide as claimed in claim 1, characterized in that it is modified compared with the native peptide and has, in particular, an alkylated group at its N-terminal end, and/or in that more amino acids are replaced with one or its/their dextrorotary form (_{D}aa), and/or in that it contains one or more peptide bonds so as to form bioisosters, for example the reduction of an amide bridge to —CH₂NH—, or a retro-inverso reaction.

4. The peptide as claimed in claim 2, in which the RGD motif is exposed via a disulfide bridge between two cysteines, in particular the peptides of sequences SEQ ID No 3 to 10:

| | |
|---|---|
| SEQ ID No 3: | GG*CRGDMFG*CGGLLRIHFRIGSRHSRIG |
| SEQ ID No 4: | GG*CRGDMFG*CGG-LFIHFRIGSRHSRIG |
| SEQ ID No 5: | GG*CRGDMFG*CGGSLFIHFRIGSRHSRIG |
| SEQ ID No 6: | GG*CRGDMFG*CGGLLFIHFKIGSRHSRIG |
| SEQ ID No 7: | GG*CRGDMFG*CGGLLFIHF^{N}RIGSRHSRIG |

(^{N}R representing an N-alkylarginine motif)

| | |
|---|---|
| SEQ ID No 8: | GG*CRGDMFG*CGGLLSRHFRIGSRHSRIG |
| SEQ ID No 9: | GG*CRGDMFG*CGGLLSIHFRIGSRHSRIG |
| SEQ ID No 10: | GG*CRGDMFG*CGGLLFRHFRIGSRHSRIG. |

5. The peptide as claimed in claim 1, characterized in that it contains a sequence

| | |
|---|---|
| SEQ ID No 11: | X-R-G-D-M-F-G-X′ |

exposing the RGD motif via a lactam bridge between the amino acids X (X)—C—O—NH—(X′), X and X′ being amino acids such that one bears an acid group and the other bears an amine.

6. The peptide as claimed in claim 5, characterized in that it corresponds to the sequences SEQ ID No 12 to SEQ ID No 23:

| | |
|---|---|
| SEQ ID No 12: | GGXRGDMFGX′GGLLFIHFRIGCRHSRIG |
| SEQ ID No 13: | GGXRGDMFGX′GGLLFIFFRIGCRFSRIG |
| SEQ ID No 14: | GGXRGDMFGX′GGLLFIHFRIGSRHSRIG |
| SEQ ID No 15: | GGXRGDMFGX′GGLLRIHFRIGSRHSRIG |
| SEQ ID No 16: | GGXRGDMFGX′GG-LFIHFRIGSRHSRIG |
| SEQ ID No 17: | GGXRGDMFGX′GGSLFIHFRIGSRHSRIG |
| SEQ ID No 18: | GGXRGDMFGX′GGLLFIHFKIGSRHSRIG |
| SEQ ID No 19: | GGXRGDMFGX′GGLLFIHF^{N}RIGSRHSRIG |

(^{N}R representing an N-alkylarginine motif)

| | |
|---|---|
| SEQ ID No 20: | GGXRGDMFGX′GGLLSRHFRIGSRHSRIG |
| SEQ ID No 21: | GGXRGDMFGX′GGLLSIHFRIGSRHSRIG |
| SEQ ID No 22: | GGXRGDMFGX′GGLLFRHFRIGSRHSRIG |
| SEQ ID No 23: | GGXRGDMFGX′GGLLFIHFRIGSRHSRIG |

7. The peptide as claimed in claim 1, characterized in that it induces apoptosis in human endothelial cells expressing αVβ3 receptors.

8. The peptide as claimed in claim 1, characterized in that it undergoes endocytosis by human endothelial cells expressing αVβ3 receptors, localizes in the mitochondrial compartment, and exerts a mitochondriotoxic effect.

9. A pharmaceutical composition, characterized in that it contains a therapeutically effective amount of at least one peptide as defined in claim 1, in combination with a pharmaceutically acceptable vehicle.

10. The pharmaceutical composition as claimed in claim 9, characterized in that it is in the pharmaceutical form suitable for its administration by injection, in particular in the form of an injectable solution for intravenous administration.

11. The use of peptides as claimed in claim 1, for producing antiangiogenic medicaments for the treatment of pathologies due to hypervascularization.

12. The use as claimed in claim 11, for producing medicaments for the treatment of solid tumors such as pulmonary tumors, adenomas, melanomas, prostate cancer, breast cancer, colon cancer, pancreatic cancer or osteosarcomas, or the treatment of diabetic retinopathies and of arthritis.
